(19) United States
(12) Patent Application Publication
(10) Pub. No.: US 2007/0060620 A1
(43) Pub. Date: Mar. 15, 2007

(54) Use of RAR retinoid agonists to increase sperm count and sperm mobility in males

(76) Inventor: John Sefton, Trabuco Canyon, CA (US)

Correspondence Address: Robert J. Baran;ALLERGAN, INC., Legal Department, 2525 Dupont Drive, Irvine, CA, 92612, US

(21) Appl. No.: 11/453,729

(22) Filed: Jun. 15, 2006

Related U.S. Application Data
(60) Provisional application No. 60/715,892, filed on Sep. 9, 2005.

(51) Int. Cl. A61K 31/4436

(52) U.S. Cl. 514/337

(57) ABSTRACT

The present invention provides a method for treating infertility which may be caused by sperm immobility, comprising administering to a person in need of treatment for infertility an effective amount of a pharmaceutical composition comprising a RAR retinoid selective compound to activate sperm motility, together with a pharmaceutically acceptable excipient, and thereby providing sperm with improved motility for treating infertility.

### CROSS REFERENCE TO RELATED APPLICATIONS

This application is based on, and claims the benefit of, U.S. Provisional Application No. 60/715,892, filed Sep. 9, 2005, and which is incorporated herein by reference.

### BACKGROUND OF THE INVENTION

1. Field of the Invention

The present invention relates to a method of improving sperm mobility by administering to a male animal, e.g. a human male, an effective amount of a RAR-selective retinoid.

2. Description of the Related Art

It is previously known that only spermatozoa with a good ability to move and with a relatively straightlined progressive motility have the possibility to achieve fertilization and that only the spermatozoa with the best morphology enter the cervical mucus (Fredricsson B. and Bjork G. (1977): Morphology of postcoital spermatozoa in the cervical secretion and its clinical significance. Fertil. Steril. 28:841-845. It is also previously known that serum support sperm motility/ Austin R. (1985) “Sperm maturation in the male and female genital tracts.” In: Biology of Fertilization, vol. 2 (eds C. Metz and A. Monroy) pp. 121-149, Academic Press, New York and is therefore used in connection with sperm separation for fertilization in vitro.

However, there are problems inherent in the use of a fluid comprising thousands of components and a specific active component would give a safer and more predictible effect than such a fluid. An active factor would also give the effect without dangerous concomittant actions e.g. immunologic factors or infectious diseases.

Commercial breeding and egg-laying operations have long sought to identify highly fecund males that would be effective breeders. A “fecund” male is one capable of producing many offspring, as opposed to a “fertile” male, which is merely capable of producing offspring. The identification of highly fecund animals is very important to the profitable operation of commercial breeding businesses. High producing males, whose sperm reliably fertilizes female eggs, improve the productivity and profitability of breeding and laying operations. A variety of techniques have been used to improve male productivity, including artificial insemination and semen evaluation.

The identification or isolation of motile sperm has been extensively studied in both human and veterinary medicine. The widespread adoption of assisted insemination, such as intrauterine insemination, gamete intrafallopian transfer (GIFT), or in vitro fertilization (IVF) for infertile human patients, has particularly stimulated the development of techniques for isolating, from an individual donor, a sub-population of sperm that has increased fertilizing capacity. One such assay involves layering a liquid medium on top of a semen sample, and allowing the sperm to swim-up into the medium. The sperm are then recovered and evaluated for concentration, motility and morphology. Alternatively, sperm for in vitro fertilization can be selected by centrifuging sperm samples through gradients of Percoll solution.

Oral retinoids currently used for the treatment of psoriasis (acitretin and etretinate) and acne (isotretinoin) are known to be associated with an increase in sperm count and sperm motility. The side effects associated with the use of these drugs are considerable and would likely preclude the use of these drugs in elevating sperm count and motility in otherwise healthy males. It is also known that retinoid antagonists are associated with decreases in sperm counts in animals, which might suggest that sperm production and motility is retinoid-dependent.

### BRIEF SUMMARY OF THE INVENTION

The present invention provides a method of improving the fertility of, and/or for improving the motility or sperm count of the sperm in semen produced by a male individual, e.g. a mammal, including but not limited to a human being, and other species such as birds and fish, comprising administering an effective amount to said individual of a RAR-selective compound, e.g. tazarotene.

In particular, it has been found that the administration of an RAR-selective retinoid compound improves fertility in both subfertile individuals as well as fertile individuals.

Accordingly, in a first aspect, the invention relates to a preparation or composition for improving the fertility of, and/or for improving the quality of the semen produced by, a male individual of a mammalian species, said composition comprising:

at least one source of a RAR retinoid selective compound, e.g. tazarotene;

and optionally;

one or more further components;

optionally one or more carriers, excipients and/or adjuvants.

The invention also relates to a method for improving the fertility of, and/or for improving the quality of the semen produced by, a male individual of a mammalian species, said method comprising administering to said male individual of a mammalian species:

at least one source of a RAR retinoid selective compound, e.g. tazarotene;

optionally one or more further components;

and more in particular in the form of a preparation or composition as described above.

In yet another aspect, the invention relates to the use of at least one source of a RAR retinoid selective compound, e.g. tazarotene in the preparation of a preparation or composition for improving the fertility of, and/or for improving the quality of the semen produced by, a male individual of a mammalian species, including but not limited to a human being.

Further aspects of the invention will become clear from the further description hereinbelow.

The preparation and/or compositions of the invention may in particular be preparations and/or compositions intended and/or suitable for oral administration to a male individual of a mammalian species, including but not limited to pharmaceutical and/or veterinary preparations or compositions.

The preparations and/or compositions may optionally contain one or more further components known per se, including but not limited to components which by themselves have some biological, pharmacological and/or veterinary activity, i.e. upon (oral) administration to a male individual of a mammalian species.

In particular, these one or more further components may be one or more compounds that are known per se to have, upon (oral) administration to a male individual of a mammalian species, a beneficial influence on the fertility and/or sperm quality of a male individual of a mammalian species; and/or may be one or more components that, upon (oral) administration to a male individual of a mammalian species, may further increase, enhance and/or facilitate the fertility- and/or semen quality-improving action of the RAR retinoid selective compound, e.g. tazarotene, that are administered according to the present invention.

The one or more further components used should most preferably be suitable for (oral) administration to a male individual of a mammalian species, and should also be compatible with the other constituents of the preparations and/or compositions of the invention, and in particular with the at least one source of RAR retinoid selective compound, e.g. tazarotene, used.

According to the invention, the source of a RAR retinoid selective compound, e.g. tazarotene, and optionally the one or more further components, are administered to a male individual of a mammalian species in an amount that, upon such (oral) administration, has a beneficial influence on the fertility of, and/or on the quality of the semen produced by, said individual, most preferably without any adverse side effects and/or health risks to said individual.

It has been found that particularly suitable amounts of administration are between 0.05 and 8 mg, and in particular 0.1 and 6 mg, per day, for the at least one source of RAR retinoid selective compound, e.g. tazarotene. The one or more further components mentioned above may be administered in suitable amounts known per se, for instance as indicated in the further description below. The amounts as described above are given for mammals having a body weight of approximately 70 kg; for mammals/vertebrates having a different body weight the above amounts should be corrected for body weight. For instance, for a mammal of 350 kg, the above amounts may be multiplied by a factor 5.

The above amounts may be administered as a single daily dose or as several doses per day. Preferably, for such administration, the source of a RAR retinoid selective compound, e.g. tazarotene, and the one or more further components are provided/administered essentially simultaneously, and in particular as (part of) a single preparation or composition.

Also, it should be noted that any administration as described above is preferably continued for a period of at least 20 days, and in particular for at least 40 days, and for example (at least) 90 days, to achieve the beneficial effect on the fertility of, and/or on the quality of the semen produced by, the male individual.

The compositions and/or preparations of the invention preferably contain the at least one source of a RAR retinoid selective compound, e.g. tazarotene, and optionally the one or more further components, in amounts that allow the above amounts to be administered, i.e. as one or more doses per day.

Thus, the preparations and/or compositions preferably contain the at least one source of a RAR retinoid selective compound, e.g. tazarotene in an amount of between 0.05 and 8 mg, and preferably of between 0.1 and 6 mg. The one or more further components may also be present in suitable amounts.

For example, such components may include folic acid and/or zinc as set forth in U.S. Pat. No. 6,576,634 or apolipoprotein and immunoglobin as set forth in U.S. Pat. No. 5,453,354 or vitamin B12, magnesium, betaine and/or 5-adenosyl methionine as set forth in U.S. Pat. No. 6,576,634.

Preferably, the at least one source of a RAR retinoid selective compound, e.g. tazarotene, and optionally the one or more further component are comprised as a unit dose.

Some particularly preferred compositions according to the present invention will be described hereinbelow.

The preparations or compositions of the invention may be in the form suitable for administration, and in particular for oral administration, to a male individual of a mammalian species, including but not limited to a human being. As such, the compositions of the invention may for instance be in the form of:

a pharmaceutical composition suitable for (oral) administration, such as a tablet, powder, capsule, sachet, syrup, elixer; and/or

a veterinary composition suitable for (oral) administration to the intended mammal.

The formulation/preparation of such preparations or compositions may be carried out in a manner known per se, while will generally comprise combining the at least one source of a RAR retinoid selective compound, e.g. tazarotene, optionally the one or more further components with each other and optionally with one or more acceptable carriers, excipients, adjuvants; depending upon the intended final preparation or composition, as will be clear to the skilled person.

The preparations and compositions of the invention may generally be used to improve the fertility of, and/or to improve the quality of the semen produced by, a male individual of a mammalian species, e.g. to improve the changes of reproduction of said male individual and/or to provide the quality of the sperm produced by said male individual.

For instance, the compositions and/or preparations of the invention may be administered to human beings; to economically important animals such as cattle, sheep, pigs and horses; to pets such as cats and dogs; to zoo animals and/or to any other desired mammal. More generally, and in additional the compositions of the invention may also be used in male individual of a non-mammal, vertebrate species, such as species of birds, fowl (i.e chicken), reptiles, and/or fish.

As such, the compositions of the invention may be administered to fertile male individuals, to sub-fertile male individuals, to male individuals which are suspected to be sub-fertile and/or to male individuals which are at (increased) risk of becoming sub-fertile, e.g. due to drug therapy, radiation therapy, disorders of the urogenital tract, and/or due to other factors, such as environmental factors and/or the further factors mentioned above.

For example, in fertile male individuals, increase of sperm counts in fertile male animals may be of interest to increase further the chance of fast reproduction, which can have an economical benefit in animal breeding, but also may have psychological and practical advantages in men.

Also, in fertile male individuals, the invention may be used to provide sperm of improved quality, e.g. for use in animal breeding programs; for use in insemination, IVF and other fertilization techniques; and/or when sperm is to be frozen and stored over longer periods of time.

In sub-fertile male individuals, the compositions of the invention may be used to treat said sub-fertility, i.e. again to enable or at least to improve the chances of successful fertilization.

In another advantageous application, the compositions and preparations of the invention may be administered to a male individual of a mammalian species, and in particular to a human, without it being known a priori whether said male individual is fertile or sub-fertile. For instance, in this application, the compositions of the invention may be administered to the male individual of a couple when fertilization is retarded, e.g. to obviate testing and/or as a first step before any further testing is carried out. This is important since sperm testing is expensive, places a burdon on the individual involved, and because the specificity and sensitivity of testing systems vary, and because there may be fluctuations in sperm count and/or sperm quality.

The compositions of the invention may be also used (i.e. prophylactically) by male individuals of couples that are intending to have a baby, i.e. to increase the chances of fertilization and/or to counteract any problems with male fertility that may be present, again without the need of prior testing.

The compositions of the invention may also be used to prevent or counteract the effects on reproduction of fluctuations of sperm count or sperm quality over time.

In yet another important aspect, the compositions and/or preparations may be used prophylactically to prevent a decrease in fertility and/or to prevent a decrease in sperm quality in individuals at risk thereof, for instance in male individuals that are (to be) subjected to drug therapy, and in to drug therapy, radiation therapy, individuals suffering from disorders of the urogenital tract, and/or other disorders that may effect fertility and/or sperm quality, such as those mentioned above.

Also, the compositions and/or preparations of the invention may for instance be used to improve fertility and/or to improve sperm quality caused by factors such as malnutrition, environmental factors, and/or exposure to harmful substances.

### DETAILED DESCRIPTION OF THE INVENTION

Retinoid compounds having RARα, RARβ and RARγ-selectivity are disclosed in U.S. Pat. Nos. 6,534,544 and 6,025,388 which are herein incorporated by reference in their entirety.

Tazarotene is an RAR-specific retinoid agonist, which is likely to show effects similar to etretinate, acitretin and isotretinoin in elevating sperm counts and sperm motility, but with significantly fewer side effects. In phase 2 studies involving patients with psoriasis and acne, tazarotene appears to have a systemic side-effect profile which is far better than those of acitretin/etretinate and isotretinoin. This may be due to the fact that tazarotene is a more specific receptor agonist (RAR) than acitretin/etretinate and isotretinoin, which can bind also to RXRs.

It is known that male infertility is a fairly common problem among couples who are trying to conceive. In a recent study (Wyshak, Int. J. Gynaecol Obstet. 2001 June; 73(3): 237-42), over 20% of 3940 respondents to a self-administered questionnaire relating to infertility were having problems in conceiving. Of these, approximately 21% were due to the fact that the male partner had a low sperm count or low sperm motility. According to Spitz et al, “Contempary Approach to the Male Infertility Evaluation”, Current Reproductive Endocrinology, Vol. 27, Number 3, September 2000, pages 487-516, approximately 6 million couples in the USA are infertile, and among these, 30% of the factors solely relate to a male problem and another 20% to both male and female. Therefore, as many as 1.5 million males in the US may have a problem with infertility.

Thus, tazarotene and other RAR-specific retinoids are used in the method of the present invention to help with male infertility that is due to low sperm counts and/or low sperm motility. Furthermore, retinoids such as tazarotene may be used in any situation in which couples are trying to conceive, where a boost to sperm production/motility could bring about an increase in the chances that a female could become pregnant. As a practical matter, since retinoids at doses likely to be effective in these circumstances are highly teratogenic, an unintended effect might arise from the transfer of the retinoid to a female in the ejaculate of the male. Although the amount expected to be transferred is minute (in the case of etretinate about 1/200,00^{th} of a single 25 mg capsule may be transferred to a female), a retinoid could be used for a period adequate to increase sperm counts and motility, followed by a washout period of a few days to a few weeks, to allow the drug in the ejaculate to be reduced to insignificant levels. For instance, tazarotene has an effective half-life of about 7-12 hours in plasma. Thus, in a period of 3 days, levels of retinoid in plasma (and also in seminal fluid) would be less than 2%, and after about one week would be less than 0.01% of the original levels. Clinical dosing with tazarotene (eg at 3-6 mg) provide levels of tazarotenic acid (the major metabolite of tazarotene) in plasma of about 75-150 ng/mL study. Thus, in 3 days, plasma levels would be expected to drop to about 2-3 ng/mL (endogenous levels of retinoid agonists such as tretinoin, isotretinoin and 4-oxo-13-cis retinoic acid may easily total over 6 ng/mL). Sperm count and motility are likely to remain elevated for some time, since sperm maturation occurs over a period of 72-74 days.

Suitable carriers, excipients and/or adjuvants for providing tazarotene-containing compositions for use in the method of this invention are known in the art and such compositions may be prepared by the skilled artisan with reference to the present specification.

Specific formulations including tazarotene are disclosed in U.S. Pat. Nos. 6,258,830 and 6,656,500, which are hereby incorporated by reference.

While particular embodiments of the invention have been described it will be understood of course that the invention is not limited thereto since many obvious modifications can be made and it is intended to include within this invention any such modifications as will fall within the scope of the appended claims.

I claim:

1. A method for treating infertility caused by sperm immobility comprising administering to a person in need of treatment for infertility an effective amount of a pharmaceutical composition comprising a RAR retinoid selective compound, which activates sperm motility, together with a pharmaceutically acceptable excipient, and thereby providing sperm with improved motility for treating infertility.

2. A method of claim 1 wherein said compound is tazarotene.

3. A method for enhancing sperm motility in vitro comprising adding a pharmaceutical composition to sperm, in vitro, wherein said pharmaceutical composition comprises a RAR retinoid selective compound which activates sperm motility, and thereby providing said sperm with enhanced motility.

4. The method of claim 3 wherein said compound is tazarotene.

5. A method for treating subfertility comprising administering to a person in need of treatment for infertility an effective amount of a pharmaceutical composition comprising a RAR retinoid selective compound together with a pharmaceutically acceptable excipient.

6. A method of claim 5 wherein said compound is tazarotene.

7. A method according to claim 5, wherein said male's subfertility is associated with at least one of drug therapy; radiation therapy; diabetes; and variocele disorders of the urogenital tract.

8. The method of claim 1 wherein said compound is administered in an amount between 0.1 and 6 mg per day.

9. The method of claim 5 wherein said compound is administered in an amount between 0.1 and 6 mg per day.

10. The method of claim 9 wherein said administration is repeated and continued for at least 20 days.

11. The method of claim 10 wherein said administering is for at least 40 days.

12. The method of claim 11 wherein said administering is for at least 90 days.

13. The method according to claim 5 wherein said male's subfertility is associated with at least one disorder selected from the group consisting of at least one of genetic disorders, hypothalamic disorders, undescended testes, testicular cancer, germ cell aplasia, malnutrition, and environmental factors.
